(19) United States
(12) Patent Application Publication
(10) Pub. No.: US 2026/0301273 A1
(43) Pub. Date: Oct. 1, 2026

(54) SYSTEM AND METHOD FOR THREE-DIMENSIONAL COMPUTED TOMOGRAPHY RECONSTRUCTION FROM X-RAYS

(71) Applicant: The Research Foundation for The State University of New York, Albany, NY (US)

(72) Inventors: Arie Kaufman, Sharon, MA (US); Gaofeng Deng, Stonybrook, NY (US)

(73) Assignee: The Research Foundation for The State University of New York, Albany, NY (US)

(21) Appl. No.: 19/695,104

(22) Filed: Jun. 2, 2026

Related U.S. Application Data
(63) Continuation of application No. PCT/US24/56343, filed on Nov. 18, 2024.
(60) Provisional application No. 63/608,385, filed on Dec. 11, 2023.

(51) Int. Cl. G06T 12/10; G06T 5/70

(52) U.S. Cl. CPC G06T 12/10; G06T 5/70; G06T 2207/10116; G06T 2211/424

(57) ABSTRACT

Systems and methods for generating a 3D volumetric image from 2D X-ray images include receiving data representing at least one X-ray image of a region of interest as input data. Volume features are extracted from the X-ray image(s) and the extracted volume features are concatenated with a noise source to generate a noisy target volume. An iterative denoising process is performed on the noisy target volume by sampling the volume with a trained conditioned diffusion model. The method then reprojects the 3D volume of the region of interest. When the 2D X-ray images include pose data, the reprojecting operating reprojects the 3D volume with the pose data.

### CROSS REFERENCE TO RELATED APPLICATIONS

The present application is a continuation of international application PCT/US24/56343 filed on Nov. 18, 2024, which claims the benefit of priority to U.S. Provisional Application Ser. No. 63/608,385, filed on Dec. 11, 2023, and titled SYSTEM AND METHOD FOR THREE-DIMENSIONAL COMPUTED TOMOGRAPHY RECONSTRUCTION FROM X-RAYS, the disclosure of which is hereby incorporated by reference in its entirety.

### FIELD OF THE INVENTION

The present invention relates to 3D visualization using 2D image data and more particularly relates to systems and methods for producing a 3D visualization using a limited number of 2D X-ray images.

### BACKGROUND OF THE DISCLOSURE

Computed Tomography (CT) has emerged as a pivotal imaging modality with a vast array of applications in fields ranging from medical diagnostics to industrial non-destructive testing. Specifically for clinical usage, CT scanners work by utilizing a rotating X-ray tube along with a series of detectors within a gantry which capture the variations in X-ray attenuation by different tissues inside the body. These diverse X-ray measurements, obtained from multiple perspectives, are then processed using tomographic reconstruction algorithms, such as filtered back-projection and iterative reconstruction, to produce 3D cross-sectional tomographic images. This ability to image internal structures at millimeter resolution has revolutionized the way physicians perceive, understand, and diagnose subjects, in a non-invasive manner.

Despite advances in 3D tomography technology, the CT imaging process exposes a substantial amount of X-ray radiation to the scanned subjects, which can be harmful to the patients and can even lead to cancer. CT Scanners are also expensive and not always widely available at a point of care. 2D X-ray radiography, on the other hand, generally offers reduced radiation exposure, wide availability, affordability, and fast and flexible screening capabilities. 2D radiographs (X-ray images, or X-rays) are widely utilized for medical diagnosis, treatment planning, and clinical follow-ups, albeit inherently limited to 2D visualization. In such 2D visualizations, however, it can be challenging for physicians to resolve ambiguities in 3D anatomical shapes and locations, especially for overlapping structures. A common practice to reduce ambiguities has been to subject patients to orthogonal pairs of X-ray images. However, this limited number of X-ray images can still only provide information limited in the two respective 2D planes.

Recently, significant progress has been made in the area of 3D image reconstruction. Notably, Neural Radiance Fields (NeRF), further described by Mildenahll et al. in the article “Nerf: Representing scenes as neural radiance fields for view synthesis,” Communications of the ACM, 65(1):99-106, 2021, has improved scene representation and the field of photo-realistic novel views synthesis from a sparse set of 2D images. The improvements from NeRF has inspired new avenues of exploration, particularly in the context of novel view synthesis from limited images. To achieve this, an image conditioned generalizable NeRF model, referred to as pixelNeRF, has been proposed by Yu et al. in the article, “pixelNeRF: Neural Radiance Fields From One or Few Images,” Proceedings of the IEEE/CVF Conference on Computer Vision and Pattern Recognition, pp. 4578-4587, 2021. whereby the NeRF is conditioned on local image features. This enables view synthesis from as few as a single image. For the specific application of 3D volume reconstruction from limited X-rays, however, directly applying deterministic methods such as pixelNeRF leads to severe over smoothness and blurriness due to the inherent ambiguity and uncertainty of the problem.

Recovering a 3D volume from a limited number of 2D X-ray images is a problem that presents significant ambiguity. This inherent ambiguity arises from the fact that multiple CT volumes can precisely match the same limited set of input X-rays, making it a challenging task to uniquely reconstruct the underlying 3D structure. To mitigate this ambiguity, traditional methods, such as deformable image registration and wavelet-based reconstruction have been explored, which attempt to capitalize on the fact that the human body anatomy is relatively well-constrained.

In addition, deep-learning approaches have been proposed to mitigate this ambiguity with Generative Adversarial Networks (GANs). However, while GANs have shown promise in improving the quality of generated images, they come with their own set of drawbacks, including mode collapse and instability in training, and the need for extensive hyperparameter tuning.

Diffusion models have recently been used in a range of applications, including producing high-quality images, capturing complex data distributions, and mitigating uncertainty. These applications leverage several desirable properties of diffusion models, such as being relatively straightforward to define and efficient to train, distribution coverage, and a stationary training objective.

Diffusion models have found several applications in generative image synthesis and have demonstrated superior performance over GANs in unconditional generation. Diffusion models have also been shown to be excellent at modeling conditional distributions of images. For example, Saharia et al. have proposed Palette, a unified framework for image-to-image translation based on conditional diffusion models, which has exhibited exceptional performance in several tasks, such as image inpainting and colorization. See “Palette: Image-to-image diffusion models,” ACM SIGGRAPH 2022 Conference Proceedings, pp. 1-10, 2022. Diffusion models are also used for image superresolution from low-resolution images.

Diffusion models have also demonstrated efficacy in 3D generation tasks. Several methodologies have been proposed in this domain. For example, DreamFusion and 3DiM utilize 2D image diffusion models for constructing 3D generative models. DreamFusion is particularly notable for its text-guided 3D generation, optimizing a NeRF from scratch. Most recently, there has been research combining diffusion models and NeRF for view synthesis from a single image. While there has been some work performed on CT reconstruction with diffusion models, this work has employed non-human-readable X-ray sinogram data from a CT scan as the input, rather than synthesizing 3D models from a limited set of conventional and readily available 2D X-ray images.

It would be beneficial to provide 3D visualization without the high dose of radiation associated with CT imaging. In this regard, it would be beneficial to provide methods to augment 2D information from a limited number of conventional 2D X-ray images to provide a 3D reconstruction of an imaged region. Preferably, such a 3D visualization would provide anatomical shape, position, and spatial relation from a limited number of 2D X-rays.

### SUMMARY OF THE DISCLOSURE

Embodiments described herein provide a system and method for three-dimensional computed tomography reconstruction from X-rays. For certain applications, the present systems and methods can reduce the time for scanning and the amount of radiation dose in CT scanning, which is beneficial in both clinical and industrial settings.

As further described herein, embodiments of the present systems and methods integrate implicit neural representation and diffusion models to address the long-standing problem of CT reconstruction from few X-rays. The systems and methods provide conditional diffusion models to address blurriness caused by the ambiguity and uncertainty arising from the use of few input images. The system and method then provide a method for CT volume representation, including a neural voxel feature field, and applies a diffusion model conditioned on the feature field to sample the CT volume.

In an exemplary embodiment, a method of generating a 3D volumetric image from 2D X-ray images is provided. The exemplary method includes using data representing at least one X-ray image of a region of interest as input data. The method includes extracting volume features from the at least one X-ray image and concatenating the extracted volume features with a noise source to generate a noisy target volume. A process of iteratively denoising the noisy target volume by sampling the volume with a trained conditioned diffusion model is applied and the method concludes with reprojecting a 3D volume of the region of interest.

In some embodiments, the 2D X-ray images include pose data and the step of reprojecting further comprises reprojecting the 3D volume with the pose data. In some embodiments, the method may further include a process of fine-tuning the reprojected 3D volume.

In an exemplary application of the present methods, the region of interest is an organ, and the method further comprises segmenting the organ volume from the 3D volume. The method may include estimating properties of an organ, such as 3D shape and organ volume. As one example, the organ is lung, and method further includes steps to provide an estimate of total lung volume. In one exemplary embodiment, total lung volume is estimated based on the number of voxels within the segmented organ volume.

The present embodiments may provide an important supplement to current X-ray and CT imaging techniques and, in some cases, provide improvements on traditional CT reconstruction techniques from X-ray images using fewer X-rays and/or faster scanning. Additionally, the system and method used as a 3D visualization enhancement of the X-ray images, which are widely used in medical planning, 3D organ shape and volume analysis, diagnosis, non-medical security checks, and industrial non-destructive inspections.

Systems, methods, and non-transitory computer-readable media are provided. In one embodiment a method of generating a 3D volumetric image from 2D X-ray images includes providing data representing at least one X-ray image of a region of interest as input data. Volume features are extracted from the at least one X-ray image and the extracted volume features are concatenated with a noise source to generate a noisy target volume. The method includes iteratively denoising the noisy target volume by sampling the volume with a trained conditioned diffusion model. The method then reprojects the 3D volume of the region of interest.

Preferably, the 2D X-ray images include pose data and the reprojecting operating further comprises reprojecting the 3D volume with the pose data. The method may further include fine tuning the reprojected 3D volume.

In some embodiments, the region of interest is an organ, and the method further comprises segmenting the organ volume from the 3D volume. In certain embodiments the method further comprises estimating organ shape from the segmented organ volume. In an example where the organ is lung, total lung volume can be estimated. For example lung volume may be estimated based on the number of voxels within the segmented organ volume.

These and other aspects and features of non-limiting embodiments of the present invention will become apparent to those skilled in the art upon review of the following description of specific non-limiting embodiments of the invention in conjunction with the accompanying drawings.

### DESCRIPTION OF THE FIGURES

Embodiments of the present disclosure are described in connection with the following FIGURES, in which:

FIG. 1 is a block diagram showing an exemplary system for generating 3D images from 2D X-ray images employing a conditional diffusion model;

FIG. 2 is a flow chart illustrating an exemplary process for performing a training phase of the present embodiments of systems and methods for generating 3D images from 2D X-ray images employing a conditional diffusion model;

FIG. 3 is a flow chart illustrating an exemplary process for performing an inference phase of the present embodiments of systems and methods for generating 3D images from 2D X-ray images employing a conditional diffusion model;

FIGS. 4A-4D are graphs depicting changes in PSNR between input X-rays and reprojections, as well as the changes in PSNR, SSIM, and LPIPS between a refined volume and ground truth during iterative refinement in accordance with the present methods;

FIGS. 5A-5F are a series of images illustrating LIDC ground truth images and reconstruction; and

FIGS. 6A-6F are a series of images illustrating an exemplary reconstruction employing the present methods.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION OF THE EMBODIMENTS

The embodiments described herein provide examples of systems and methods for three-dimensional computed tomography reconstruction from a limited set of conventional X-ray images. The presently disclosed systems and methods provide for 3D visualization from 2D X-ray images which may be useful when conventional CT scanning is not available and can reduce the time for scanning and the amount of radiation dose compared to CT scanning, which can be beneficial in certain clinical and industrial settings.

Since in the context of clinical practice, it is typical for only one or two 2D X-ray images to be taken for a patient with a conventional X-ray machine, the ability to construct 3D volumes from a small number, e.g., as few as 1 or 2 X-ray images, provides clinical advantages. Such a construction, however, involves a considerable degree of ambiguity since given a significantly limited number of X-rays, multiple potential CT volumes can precisely match them. To address this problem, the present embodiments preferably apply a conditional diffusion model to reduce the ambiguity and uncertainty. When integrated with volume features extracted from the input images, the present diffusion model is 3D aware and can sample a possible 3D volume from the distribution conditioned on the input X-rays. This method improves the fidelity of the reconstructed volume and reduces the blurriness typically exhibited in the non-generative regression-based method. The present embodiments offer 3D volumetric information to X-ray images while also preserving their original 2D information. To achieve this, an iterative refinement method to enforce the consistency between the inputs and reprojections can be employed.

### Diffusion Models

Diffusion models, or diffusion probabilistic models, which are preferably used in the present embodiments, are a class of latent variable generative models used in machine learning systems. A diffusion model typically consists of three major components: the forward process, the reverse process, and the sampling procedure. The goal of a diffusion model is to learn a diffusion process that generates a probability distribution for a given dataset from which new images can be sampled. Diffusion models learn the latent structure of a dataset by modeling the way in which data points diffuse through their latent space. Diffusion models are typically formulated as Markov chains and trained using variational inference.

Diffusion models can be conditioned to generate an output based on an imposed condition rather than the whole distribution of input data. For example, a diffusion model trained on a broad corpus of images would typically generate images that look like a random image from that corpus. To generate more specific images, a condition may be imposed, such as defining a category. Conditioning typically requires converting the conditioning parameters into a vector of floating-point numbers which is applied to the underlying diffusion model neural network.

The present systems and methods include a training phase and an inference phase. During the training phase, a set of data comprising input X-ray image and CT scan pairs is used to extract the volume features from the input X-rays. During training, the volume features are concatenated with noisy CT scans which are then denoised with a conditional diffusion model. During the inference phase, a CT volume is sampled with a diffusion model conditioned on the volume features extracted from the input X-rays. To further increase the consistency between the input and the reconstruction, an iterative refinement method can be used to minimize the distance between the input X-rays and resulting 2D reprojections.

Diffusion models sample from a distribution by reversing a forward diffusion process that gradually adds noise to the data. The sampling process typically starts with Gaussian noise and produces gradually less noisy samples until reaching a final sample. Conditional diffusion models make the denoising process conditional on the input signal in the form of p(y|x).

The problem of CT reconstruction from X-rays with conditional diffusion models can

$$\{ {x}_{i , \to {y}_{i}} \} \begin{matrix}N \\ i = 1\end{matrix} \{ xi , yi \}$$

be modeled as follows. Given a dataset where x are input X-rays and y are CT scans, the aim is to learn an approximation to the conditional distribution p(y|x) of target CT scans given the input X-rays. In the present embodiments, this problem can be preferably addressed by adapting denoising diffusion probabilistic models (DDPMs) for CT image generation conditional on input X-rays.

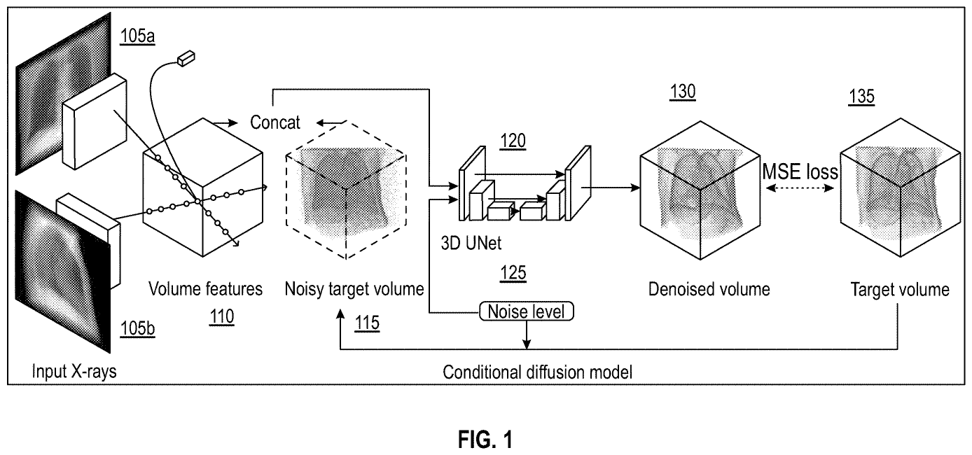

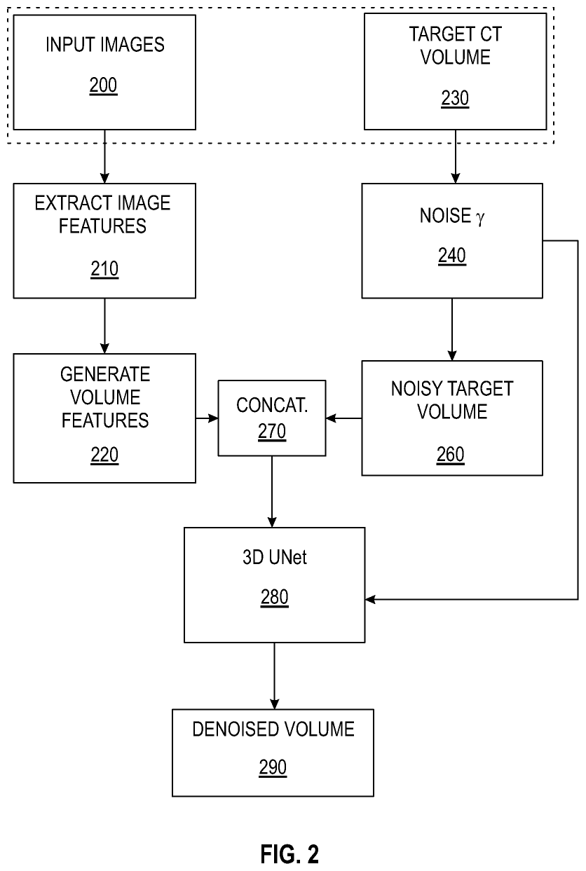

FIG. 1 is a simplified block diagram illustrating an exemplary model architecture and FIG. 2 is a simplified flow diagram illustrating exemplary steps in a training phase of the present method. Referring to FIG. 1, X-Ray images 105a, 105b are inputs to the system and volume features 110 are extracted from these images. For the paired target volume y 135 corresponding to the input X-rays x 105a, 105b in the training set, noise is added according to the noise levels γ 125 to generate the noisy volumes y 115. The noisy volume is then concatenated with volume features (FIG. 2, step 270) to generate a concatenated 3D volume. A 3D UNet U 120 can then be applied to the concatenated 3D volume to generate a denoised volume 130 given the concatenated volume as well as the noise level 125. A U-Net is a known convolutional neural network that was developed for biomedical image segmentation. The network is based on a fully convolutional neural network whose architecture was adapted to work with fewer training images and to yield more precise segmentation. The U-Net architecture which now underlies many modern image generation models has also been employed in diffusion models for iterative image denoising.

Referring to the flow diagram of FIG. 2, X-ray images are provided as input (step 200). In a preferred example, two orthogonal X-ray images are used but it will be appreciated that more than two images can be used and in some cases only a single image can be used. It will be appreciated, however, that using a lower number of images as input inherently increases the potential ambiguity in the reconstruction. Preferably, the pose of the X-ray in the respective X-rays are known and are also input to the system, such as with metadata associated with the X-ray image data.

Still referring to FIG. 2, for each input X-ray image 200, an image encoder E is used to extract image features (step 210). Given the input X-ray images x with known poses, local image features can be backprojected to the corresponding voxels to obtain volume features F, where each voxel is associated with a feature vector (step 220). Preferably, for each given voxel, the method projects the voxel onto the image plane coordinate and bilinearly interpolates the image feature volume to obtain the feature vector. The bilinearly interpolated local image feature can then be used as the voxel feature.

One potential challenge with this representation is that all voxels along the same ray will have the same voxel feature. Previously, to differentiate among these voxels, prior methods, such as applied in pixelNeRF, have concatenated the voxel feature with positional encoding of the voxel's position in the input view coordinate system. Positional encoding has a disadvantage in that it generally requires either a large deep multilayer perceptron (MLP) or extra encoding parameters with a relatively small MLP to learn meaningful representation from the coordinates. An advantage of the present methods using a diffusion model is that removing the positional encoding, which would typically be detrimental to reconstruction results for the deterministic image conditioned method, doesn't substantially adversely affect the reconstruction in the present methods since the current diffusion model naturally addresses the ambiguity. Thus, in the present methods it is possible to only use the image features to represent the voxel feature.

To incorporate voxel features extracted from two (2) or more input X-ray images, a multilayer perceptron (MLP) f can be applied to all the voxels with an average pooling layer to aggregate the voxel features from two X-rays, such as following the methodology used in pixelNeRF, to obtain the volume features F=f(E(x)) 220. Then, each CT volume of shape h×w×c is represented as volume features of shape h×w×c×d, where each voxel is associated with a feature vector of length d.

The present method can also be applied with a single X-ray as input. Specifically, after bilinearly interpolating the image feature to obtain a feature vector for each voxel from a single X-ray, the MLP f is directly applied to all the voxel features without average pooling aggregation.

Training of the models used in the present embodiments is further described in connection with the block diagram of FIG. 1 and flow diagram of FIG. 2. As illustrated in FIG. 1, training is performed using a large dataset comprising X-ray image 200 and CT scan 230 pair data. Referring to FIG. 2, input X-ray images are applied to an image encoder which extracts image features 210 an generates volume features 220, as discussed above. During a training phase, at each iteration the process randomly samples the X-ray 200 and CT scan 230 pair (x, y) from the training set and added noises of random noise levels γ 240 to the CT scan y following denoising diffusion probabilistic models (DDPMs). This can be performed as described by Ho et al, in “Denoising Diffusion Probabilistic Models,” Advances in Neural Information Processing Systems, 33:6840-6851, 2020. The noisy volume y 260 is concatenated 270 with volume features which is applied to a 3D UNet 280.

The UNet U 280 is used to denoise the noisy volume, y, given the volume features 220, F and noise level γ, to generate the denoised CT volume U (F, \tilde{y}, γ). The model is trained end-to-end to optimize E, f and U by minimizing the following loss function:

$$L = {𝔼}_{( x , y )} {𝔼}_{ε \sim 𝒩 ( 0 , I )} {𝔼}_{γ} {‖ U ( F , \tilde{y} , γ ) - y ‖}^{2} \qquad ( 1 )$$

where

$$F = f ( E ( x ) ) \qquad ( 2 )$$

and

$$\tilde{y} = \sqrt{γ y} + \sqrt{1 - γ ε} \qquad ( 3 )$$

Unlike known GAN-based CT reconstruction work, with three or more terms in the loss function, the loss function of the present embodiments shown in Equation (1) has only one mean square error (MSE) term. The simplicity of this loss function makes training easier and greatly reduces the work of hyperparameter tuning for balancing the different terms in the loss function.

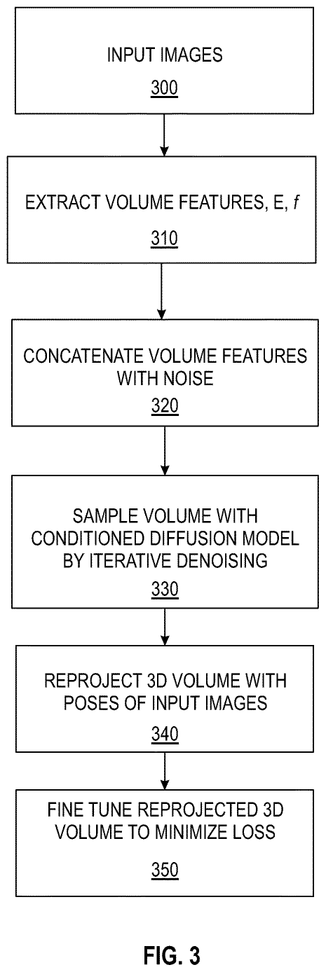

FIG. 3 is a flow diagram illustrating an inference phase of a system in accordance with the present disclosure for generating a 3D volume from a limited number of X-ray images. During an inference phase, the trained diffusion model is conditioned with the input images in order to sample the volume and generate a 3D model. Referring to FIG. 3, one or more X-ray images are input to the system in step 300. Volume features are then extracted with image encoder E and f from the input images in step 310. Then the volume features are concatenated with noise randomly drawn from a 3D Gaussian distribution in step 320. The present methods then sample the volume with the diffusion model conditioned on the concatenation of volume features and 3D Gaussian noise by iterative denoising of the noisy volume in step 330. Preferably, denoising diffusion implicit models (DDIMs) can be used for faster sampling, such as disclosed by J. Song et al. in “Denoising Diffusion Implicit Models,” arXiv preprint arXiv: 2010.02502, 2020. In one embodiment, 25 ddim denoising steps have been found suitable as a default setting, but other denoising steps may also be used.

Diffusion models generate images with an iterative denoising process starting from Gaussian noise. As a result, the number of denoising steps plays an important role in both the generated image quality as well as the computational cost. In this regard, a lower number of sampling steps typically leads to more blurry and smoother images, while more sampling steps result in more details in the reconstruction with higher associated computational cost. This can also be seen in Table 1 below, where the present model was evaluated on the test set with different denoising steps. It shows that when the number of steps increases, PSNR and SSIM (which favor over-smoothness and blurriness) also decrease while the performance on LPIPS increases (lower LPIPS means higher similarity).

TABLE 1
Effect of the number of Denoising Steps

| Steps | PSNR ↑ | SSIM ↑ | LPIPS ↓ |
|---|---|---|---|
| 2 | 23.161(±1.130) | 0.698(±0.048) | 0.185(±0.034) |
| 5 | 22.841(±1.068) | 0.697(±0.048) | 0.163(±0.031) |
| 10 | 22.629(±1.032) | 0.690(±0.048) | 0.147(±0.029) |
| 15 | 22.540(±1.029) | 0.686(±0.049) | 0.141(±0.028) |
| 25 | 22.434(±1.010) | 0.680(±0.050) | 0.135(±0.027) |

### Iterative Refinement

The present embodiments can sample a CT volume from the volume features extracted from the input X-ray images in a feed-forward manner. An objective is not only to offer 3D information but also to preserve the original 2D information from input X-rays. To achieve this, a method to iteratively refine the initial reconstructed 3D CT volume by enforcing the consistency between the input X-rays and reprojections can be used which perseveres more information in the original X-rays, step 330.

Specifically, given the input X-rays with known poses, the local image features are projected to the corresponding voxels and obtain the volume features, which are concatenated with a noise source, such as a substantially pure 3D Gaussian noise. The model of the present embodiments generates an initial 3D volume from the concatenated volume. The initial 3D volume is then re-projected with the poses of input X-rays to generate the reprojections, step 340. The process may preferably include a process to fine-tune the model to refine the 3D reconstruction by minimizing the L2 loss between the input X-rays and reprojections, step 350. During the fine-tuning, the initial 3D Gaussian noise and UNet U are fixed and fine-tuning is performed for the image encoder E and MLP f.

Fine tuning can help the model find the volume features generating the 3D CT that best matches the input X-rays. Gradient propagation through the sampling process of diffusion models is typically computationally expensive, due to the iterative sampling. Gradient checkpointing can be used to reduce the memory cost with the cost of increased computation time. While resulting in more computation, this can substantially improve the reconstruction quality.

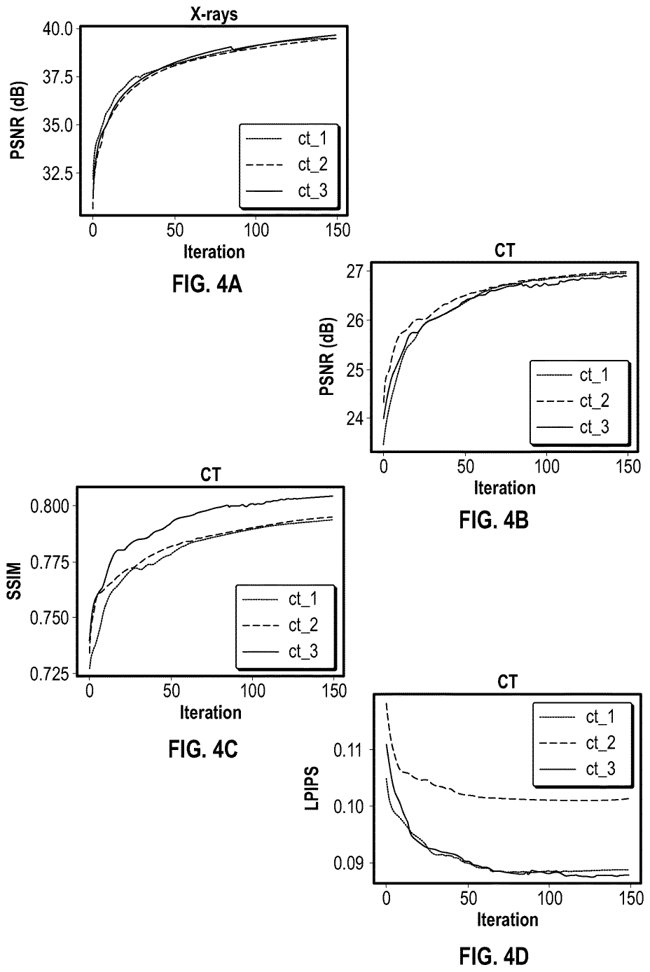

FIGS. 4A-4D are graphs illustrating changes in PSNR between input X-rays and reprojections, as well as the changes in PSNR, SSIM, and LPIPS between the refined volume and ground truth during iterative refinement of 3 CT volumes with 150 iterations at 25 DDIM denoising steps. During the refinement process, the consistency between input X-rays and reprojections is significantly improved. Also, all three metrics are also substantially improved.

While iterative refinement enhances consistency between the inputs and reprojections, as well as the 3D reconstruction quality, it also increases computational demands. This is particularly noticeable in diffusion models due to the iterative sampling. To reduce the refinement time, multiple different sized denoising steps can be applied. For example, two different denoising step sizes can be used for iterative refinement. In an example with 100 iterations, the first 99 iterations can use two (2) denoising steps and twenty-five (25) steps (ddim25) used for the final iteration, since ddim2 is faster but generates lower quality image quality while ddim25 generates better image quality but is more resource intensive.

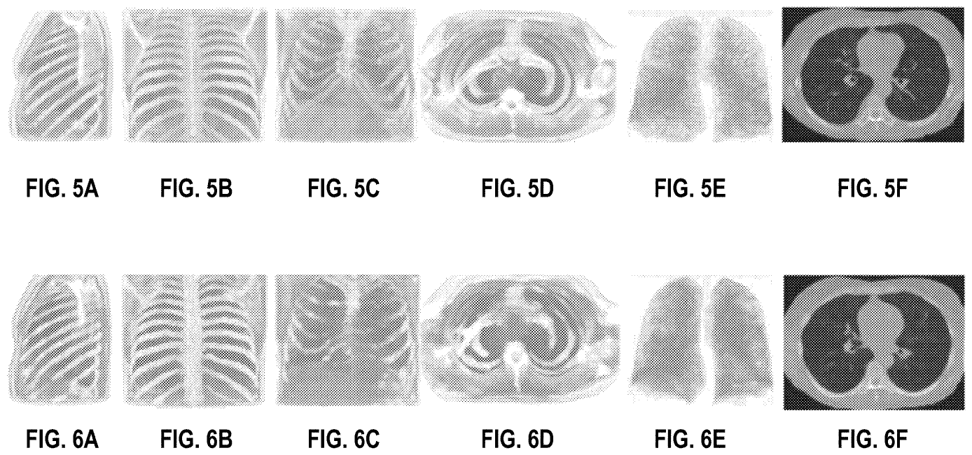

FIGS. 5A-5F are a series of images, including side (FIG. 5A), front (FIG. 5B), and rear 2D views (FIG. 5C), cross sectional views (FIGS. 5D, 5F) and a 3D reconstruction of LIDC ground truth data (FIG. 5D). FIGS. 6A-6F present the corresponding image views as FIGS. 5A-5F showing a qualitative comparison of a reconstruction using the present methods with two orthogonal images used as input data, compared to the ground truth of FIGS. 5A-5F. As can be seen in FIGS. 6A-6F, the present reconstruction methods allow the reconstruction of a 3D volume to augment the 2D information. This enables assessment of and visualization of 3D information from a small set of 2D X-rays. With the application of different transfer functions, the 3D shape and position of different organs and bones can be visualized.

### Evaluation of 3D Organ Shape Reconstruction:

The present systems and methods provide for an effective evaluation of 3D organ shape. The reconstruction of 3D anatomical shape from a limited number of 2D X-rays has been applied in various medical applications, including visualization of lung motion during respiration, hip replacement planning, and risk assessment of osteoporosis. The present systems and methods can be used to visualize 3D anatomical information such as 3D lung shape and body shape from limited number of 2D X-rays. Using 3D lung shape as an illustrative example, the present method first segments the lung regions from the CT volumes. Lung masks are obtained by segmenting the lung regions from both the ground truth CT volumes in the test set and the corresponding reconstructed CT volumes. This can be performed, for example, by using the lung segmentation pipeline described in the article “Evaluate the malignancy of pulmonary nodules using the 3-d deep leaky noisy- or network,” by Liao et al, IEEE Transactions on Neural Networks and Learning Systems, vol. 30, no. 11, pp. 3484-3495, 2019. It will be appreciated that other segmentation methods may also be used to isolate the region of interest of a particular organ from surrounding voxels.

The segmentation masks may be visually inspected and cases which do not adequately identify the volume of interest can be manually removed. To evaluate the similarity between the reconstructed and the ground truth 3D lung shapes, the Dice Similarity Coefficient (Dice) and Intersection over Union (IoU) between the 3D lung masks of ground truth volumes and reconstructed volumes can be calculated. In one example, a 3D lung shape was extracted using the lung segmentation masks from the 3D volume using the present methods with an average Dice similarity over 0.92 without refinement and over 0.95 with refinement.

### Total Lung Volume Estimation

With the ability to estimate organ contours in 3D space, the present methods further open the door to other computer-aided diagnostic tools. For example, total lung volume (TLV) is an important quantitative biomarker for assessing the severity, progression, and treatment response in obstructive and restrictive lung diseases. Specific patterns of temporal changes in TLV can be identified in patients with these diseases. TLV is also significant in procedures such as lung volume reduction surgery and lung transplant. A commonly used method for measuring TLV is the pulmonary function test (PFT) with special techniques such as gas dilution (usually with helium) or whole-body plethysmography. Several studies demonstrated that TLV calculated from CT is strongly correlated with TLV measured from PET. CT-derived TLV is used in various medical conditions, including the assessment of chronic obstructive pulmonary disease (COPD) and restrictive lung disease, as well as in lung volume reduction surgery and lung transplant. However, as in other applications, using CT scans to evaluate TLV has practical limitations and challenges, such as radiation exposure and high costs. In contrast, conventional chest X-rays are simpler, faster, more accessible, and expose patients to lower radiation.

The present methods of 3D reconstruction from 2D X-ray images can be applied to estimate TLV by measuring TLV in the present 3D CT volumes reconstructed from 2D chest X-rays. The lung volume for each CT reconstruction can be calculated by multiplying the CT voxel size by the number of voxels in the segmented lung masks. Mean absolute error (MAE), mean absolute percentage error (MAPE), and Pearson correlation coefficient (r) can be computed to demonstrate the relationship between the estimated lung volumes from reconstructed CT scans and the reference lung volumes from ground truth CT scans. The present methods can be used to estimate TLV with MAPE below 3%. With iterative refinement, the error percentage is reduced to below 1%.

### Paired Dataset Generation

The training of a deep learning model for 3D CT reconstruction from X-rays preferably uses a large dataset of paired X-rays and their corresponding 3D CT reconstruction. However, creating such a large paired dataset can be expensive and currently no suitable public paired dataset is available in the medical domain. Thus, as an alternative to using paired datasets generated from actual patient data for both the X-Ray and CT image data, synthetic paired data can be generated for training using actual CT scans and generating synthetic X-ray images from those scans to get the dataset of paired X-rays and CT scans. Specifically, digitally reconstructed radiographs (DRRs) of desired views can be synthesized using Siddon's ray tracing algorithm, such as disclosed by Siddon et al in “Fast Calculation of the Exact Radiological Path for Three-Dimensional CT Array,” Medical physics, 12(2):252-255, 1985. In this way, a large dataset of paired synthetic X-rays (DRRs) and real CT scans is developed to train the deep learning model.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium or computer-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random-access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A method of generating a 3D volumetric image from 2D X-ray images, comprising:
input data representing at least one X-ray image of a region of interest;
extract volume features from the at least one X-ray image data;
concatenate the extracted volume features with a noise source to generate a noisy target volume;
iteratively denoise the noisy target volume by sampling the volume with a trained conditioned diffusion model; and
reproject a 3D volume of the region of interest.

2. The method of generating a 3D volumetric image of claim 1, wherein the 2D X-ray image data includes pose data and the reprojecting step further comprises reprojecting the 3D volume with the pose data.

3. The method of generating a 3D volumetric image of claim 1, further comprising fine tuning reprojected 3D volume.

4. The method of generating a 3D volumetric image of claim 1, wherein the region of interest is an organ, and the method further comprises segmenting the organ volume from the 3D volume.

5. The method of generating a 3D volumetric image of claim 4, further comprising estimating organ shape from the segmented organ volume.

6. The method of generating a 3D volumetric image of claim 4, wherein the organ is lung, and wherein total lung volume is estimated based on the number of voxels within the segmented organ volume.

7. A system for generating 3D volumetric image from 2D X-ray images, comprising:
an image encoder receiving data representing at least one 2D X-ray image, extracting image features and generating volume-based voxel features therefrom;
a noise source generating a noisy target volume;
a conditional diffusion model trained on X-ray image-CT volume pair data, the diffusion model:
receiving the voxel features from the image encoder and the noisy target volume;
concatenating the voxel features with the noisy target volume and iteratively denoising the noisy target volume; and
reprojecting the denoised volume as a 3D volume of the region of interest.

8. The system of claim 6, wherein the 2D X-ray image data includes pose data and reprojecting further comprises reprojecting the 3D volume with the pose data.

9. The system of claim 6, further comprising a processing step of fine tuning reprojected 3D volume.

10. The system of claim 6, wherein the region of interest is an organ, and the system further comprises a processor segmenting the organ volume from the 3D volume.

11. The system of claim 10, further comprising a process to visualize the estimated shape of the organ from the segmented organ volume.

12. The system claim 10, wherein the organ is lung, and wherein total lung volume is estimated based on the number of voxels within the segmented organ volume.

13. A non-transitory computer readable media programmed with instructions for a processor to perform method of generating a 3D volumetric image from 2D X-ray images, comprising:
input data representing at least one X-ray image of a region of interest;
extract volume features from the at least one X-ray image data;
concatenate the extracted volume features with a noise source to generate a noisy target volume;
iteratively denoise the noisy target volume by sampling the volume with a trained conditioned diffusion model; and
reproject 3D volume of the region of interest.

14. The non-transitory computer readable medium of claim 11, wherein the 2D X-ray image data includes pose data and reprojecting further comprises reprojecting the 3D volume with the pose data.

15. The non-transitory computer readable medium of claim 11, wherein the method of generating a 3D volumetric image further comprises fine tuning reprojected 3D volume.

16. The non-transitory computer readable medium of claim 11, wherein the region of interest is an organ, and the method further comprises segmenting the organ volume from the 3D volume.

17. The non-transitory computer readable medium of claim 16, further comprising the step of estimating organ shape from the segmented organ volume.

18. The non-transitory computer readable medium of claim 16, wherein the organ is lung, and wherein total lung volume is estimated based on the number of voxels within the segmented organ volume.
